(19) United States
(12) Patent Application Publication
(10) Pub. No.: US 2018/0344899 A1
(43) Pub. Date: Dec. 6, 2018

(54) ENHANCEMENT OF NUCLEUS PULPOSUS REGENERATION BY ENHANCED PERFUSION OF PERISPINAL AREA BY COMBINATION DRUG, GENE AND CELLULAR THERAPIES

(71) Applicant: Creative Medical Technologies, Inc., Phoenix, AZ (US)

(72) Inventors: Thomas Ichim, San Diego, CA (US); Amit Patel, Salt Lake City, UT (US); Donald Dickerson, Del Mar, CA (US)

(73) Assignee: Creative Medical Technologies, Inc., Phoenix, AZ (US)

(21) Appl. No.: 15/996,182

(22) Filed: Jun. 1, 2018

Related U.S. Application Data
(60) Provisional application No. 62/513,670, filed on Jun. 1, 2017.

(51) Int. Cl. A61L 27/38; A61K 35/28; A61K 35/545; A61L 27/36

(52) U.S. Cl. CPC A61L 27/3834; A61K 35/28; A61K 38/19; A61L 27/3641; A61K 35/545

(57) ABSTRACT

Disclosed are methods of enhancing regeneration of the nucleus pulposus through enhancement of perispinal perfusion in combination with a regenerative intervention. In one embodiment the invention teaches stimulation of enhancement of perispinal perfusion by administration of angiogenic agents prior to performing a regenerative intervention such as administration of a stem cell therapy. In one specific embodiment perispinal angiogenesis is stimulated by administration of autologous bone marrow mononuclear cells in the perispinal area, preferably into a muscular area, subsequent to which intradiscal administration of regenerative cells, such as mesenchymal stem cells is performed.

### CROSS-REFERENCE TO RELATED APPLICATIONS

This application claims benefit of priority to U.S. Provisional Application No. 62/513,670, filed Jun. 1, 2017, which is hereby incorporated by reference in its entirety.

### FIELD OF THE INVENTION

The present invention relates generally to materials and methods for augmenting and/or repairing intervertebral discs, and more particularly to materials and methods for augmenting and/or repairing intervertebral discs with stem cell material, more particularly, the invention relates to augmentation of stem cell therapeutics for treatment/regeneration of nucleus pulposus material by enhancement of perfusion and augmenting the microenvironment.

### BACKGROUND OF THE INVENTION

The healthy intervertebral disc facilitates motion between pairs of vertebrae while absorbing and distributing shocks. The disc is composed of two parts: a soft central core (the nucleus pulposus) that bears the majority of the load, and a tough outer ring (the annulus fibrosis) that holds and stabilizes the core material. As the natural aging process progresses, the disc may dehydrate and degenerate, adversely affecting its ability to adequately cushion and support the vertebral bodies. This natural desiccation, which in its more advanced state is often referred to as “black disc” because of the disc's dehydrated appearance on Magnetic Resonance Imaging [MRI], can cause discomfort to the patient as the vertebrae to come closer together—compressing the spinal nerves and causing pain.

Techniques for addressing degenerative disc disease have heretofore relied primarily on disc replacement methods. In cases in which a dehydrated and/or degenerating disc was augmented before disc replacement was required, the augmentation materials have primarily been synthetic devices that expand, are inflated, or deploy expanding elements when implanted into the disc.

Pluripotent and/or multipotent stem cells have been suggested as being potentially useful for medical applications. Pluripotent stem cells are self-renewing cells which are capable of differentiating into any one of more than 200 different cell types found in the body. Embryonic pluripotent and/or multipotent stem cells may be characterized as either embryonal carcinoma (“EC”) cells, embryonic germ (“EG”) cells, or embryonic stem (“ES”) cells. Non-embryonic pluripotent and/or multipotent stem cells may be obtained from adult somatic cell sources. Non-embryonic multipotent stem cells include, for example, neural stem cells, mesenchymal stem cells, bone marrow stem cells, and stem cells obtained from liposuction. For the purposes of this disclosure, embryonic pluripotent or multipotent cells, and non-embryonic pluripotent or multipotent cells, are all referred to as “stem cells.” In other words, any cell that has not differentiated into a mature cell type, may be referred to as a “stem cell” for the purposes of this disclosure.

Mesenchymal stem cells are adult multipotent cells derived from multiple sources, including bone marrow stroma, blood, dermis, and periosteum. These cells can be cultured continuously in vitro without spontaneous differentiation. However, under the proper conditions, mesenchymal stem cells can be induced to differentiate into cells of the mesenchymal lineage, including adipocytes, chondrocytes, osteocytes, tenocytes, ligamentogenic cells, myogenic cells, bone marrow stroma cells, and dermogenic cells.

Hematopoietic stem cells are multipotent cells capable of self renewal and differentiation into multiple blood cells types, including erythrocytes, megakaryocytes, monocytes/macrophages, granulocytes, mast cells, B-cells and T-cells. Hematopoietic stem cells can be obtained from fetal liver, adult bone marrow, or mononuclear muscle precursor cells called satellite cells.

Among the literature references relating to the use of stem cells to grow or treat tissue are U.S. patent Publications Nos. 2002/0076400, 2003/0054331, and 2004/0171146, all of which provide background to the present invention.

Unfortunately in conditions of disc degeneration a perfusion defect exists in patients that does not allow for regeneration of nucleus pulposus because of metabolite build up. The current patent addresses this issue.

### SUMMARY

Embodiments herein are directed to methods of augmenting regeneration of the nucleus pulposus comprising the steps of: a) identifying a patient with lower back pain; b) assessing perfusion rate of perispinal area of said patient; c) selecting patients possessing a perfusion defect in said lower back; d) administering to said patients an angiogenesis stimulating treatment in the perispinal area at a concentration and frequency sufficient to enhance local perfusion; and e) administering a regenerative intervention intradiscally to the nucleus pulposus.

### DETAILED DESCRIPTION OF THE INVENTION

The main cause of chronic low back pain, that is, intervertebral disc degeneration, is an irreversible change. Since 1993, the present inventors have been experimentally proving the effect of reinsertion of activated nucleus pulposus in the suppression of intervertebral disc degeneration for the purpose of suppressing the intervertebral disc degeneration and regenerating intervertebral discs and have begun the clinical application thereof. However, there is a limit to the number of cells which can be collected from degenerated intervertebral discs. The only option is to collect fresh nucleus pulposus from healthy intervertebral discs, but the collection of fresh nucleus pulposus has been considered to be difficult in practice. Therefore, the present inventors attempted to regenerate intervertebral discs using mesenchymal stem cells, or multipotent stem cells having complete plasticity, and confirmed a certain effect in animal experiments, whereby the present invention has been completed.

The present inventors found that augmentation of perfusion in the perispinal area enhances the efficacy of mesenchymal stem cells or multipotent stem cells having complete plasticity transplanted into a degenerated intervertebral disc are later induced to intervertebral disc-like or intervertebral disc cells themselves, whereby the intervertebral discs are regenerated in function.

One aspect of the present invention, there is provided a method of augmenting and/or repairing an intervertebral disc by administering stem cell material into the disc subsequent to enhancement of perispinal perfusion by stimulation of angiogenesis. Stimulation of angiogenesis is accomplished by administration of angiogenic factors and/or cells producing angiogenic factors in the perispinal area, preferably into the muscles associated with the perispinal area.

Some studies showed that the vast majority of patients with long-term lower back pain often have occluded lumbar/middle sacral arteries and that occlusion of these arteries is associated with disc degeneration [5]. Furthermore, patients with high LDL cholesterol complained of more severe back symptoms than those with normal value [5]. These findings support previous studies that occlusion of lumbar/middle sacral arteries is associated with lower back pain and disc degeneration [6-10] and that occlusion of these arteries is due to atherosclerosis [11, 12]. Epidemiologic and postmortem studies indicate that atheromatous lesions in the abdominal aorta may be related to disc degeneration and long-term back symptoms [6-10]. The blood supply of the lumbar spine is/derived from the aorta through the lumbar and middle sacral arteries. The upper four segments of the lumbar spine receive their blood supply from the four pairs of the lumbar arteries, which arise in the posterior wall of the abdominal aorta. The fifth lumbar segment is supplied partly by the middle sacral artery (arising in the bifurcation) and partly by branches of the iliolumbar arteries (arising from the internal iliac arteries) [13, 14]. Nutrition of the avascular intervertebral disc occurs by diffusion through the vertebral endplates from the blood vessels in the vertebral bodies above and below the disc [15, 16]. Cholesterol plaques in the wall of the aorta obliterate orifices of lumbar and middle sacral arteries and decrease blood supply of the lumbar spine and its surrounding structures. As a result, structures with precarious nutrient supply, such as the intervertebral discs, gradually degenerate [11, 17, 18]. Reduced blood flow causes hypoxia and tissue dysfunction. It also hampers removal of waste products, such as lactic acid. These changes in turn may irritate nociceptive nerve endings, causing pain, as well as lead to deterioration and atrophy of the structures involved [19-22]. Stimulation of local angiogenesis is aimed to overcome these perfusion defects, thus allowing for ability to stem cells or regenerative cells injected to function optimally.

In one embodiment, angiogenesis is stimulated in the perispinal area by administration of platelet rich plasma alone or in combination with cells to augment regional perfusion. In some embodiments, stem cells are administered together with platelet rich plasma for stimulation of angiogenesis. Said stem cells are cultured with cytokines, growth factors, peptides, or combinations prior to administration. In another embodiment the disclosure teaches augmentation of angiogenic regenerative activities through prior culture with platelet rich plasma (PRP). In another embodiment the disclosure provides means of co-administering factors or PRP together with stem cells for therapeutic activity enhancement.

It is known that there are growth factors, cytokines and peptides released from activated platelets, one strategy to therapeutically leverage this fact is to prepare an autologous platelet concentrate suspended in plasma, also known as PRP. Several means of preparing PRP are known in the art that are useful for the practice of the invention, some of which are described in the following and incorporated by reference [23, 24]. Examples of devices used for generation of PRP include SmartPReP, 3iPCCS, Sequestra, Secquire, CATS, Interpore Cross, Biomet GPS, and Harvest's BMAC [25]. Other means of generating PRP are described in U.S. Pat. Nos. 5,585,007, 5,599,558, 5,614,204, 6,214,338; 6,010,627; 5,165,928; 6,303,112; 6,649,072, 6,649,072, which are incorporated by reference herein in their entirety.

Subsequent to stimulation of angiogenesis, stem cells that are injected into the nucleus pulposus are defined as stem cells or “enhanced” stem cells. Said stem cells are mesenchymal, hematopoietic, or pluripotent.

Mesenchymal stem cells (“MSC”) were originally derived from the embryonal mesoderm and subsequently have been isolated from adult bone marrow and other adult tissues. They can be differentiated to form muscle, bone, cartilage, fat, marrow stroma, and tendon. Mesoderm also differentiates into visceral mesoderm which can give rise to cardiac muscle, smooth muscle, or blood islands consisting of endothelium and hematopoietic progenitor cells. The differentiation potential of the mesenchymal stem cells that have been described thus far is limited to cells of mesenchymal origin, including the best characterized mesenchymal stem cell (See Pittenger, et al. Science (1999) 284: 143-147 and U.S. Pat. No. 5,827,740 (SH2.sup.+SH4.sup.+CD29.sup.+CD44.sup.+CD71.sup.+CD90.sup.+CD106.sup.+CD120a.sup.+CD124.sup.+CD14.sup.−CD34.sup.−CD45.sup.−)). The invention teaches the use of various mesenchymal stem cells

In one embodiment MSC donor lots are generated from umbilical cord tissue. Means of generating umbilical cord tissue MSC have been previously published and are incorporated by reference [1-7]. The term “umbilical tissue derived cells (UTC)” refers, for example, to cells as described in U.S. Pat. No. 7,510,873, U.S. Pat. No. 7,413,734, U.S. Pat. No. 7,524,489, and U.S. Pat. No. 7,560,276. The UTC can be of any mammalian origin e.g. human, rat, primate, porcine and the like. In one embodiment of the invention, the UTC are derived from human umbilicus. umbilicus-derived cells, which relative to a human cell that is a fibroblast, a mesenchymal stem cell, or an iliac crest bone marrow cell, have reduced expression of genes for one or more of: short stature homeobox 2; heat shock 27 kDa protein 2; chemokine (C—X—C motif) ligand 12 (stromal cell-derived factor 1); elastin (supravalvular aortic stenosis, Williams-Beuren syndrome); Homo sapiens mRNA; cDNA DKFZp586M2022 (from clone DKFZp586M2022); mesenchyme homeobox 2 (growth arrest-specific homeobox); sine oculis homeobox homolog 1 (Drosophila); crystallin, alpha B; disheveled associated activator of morphogenesis 2; DKFZP586B2420 protein; similar to neuralin 1; tetranectin (plasminogen binding protein); src homology three (SH3) and cysteine rich domain; cholesterol 25-hydroxylase; runt-related transcription factor 3; interleukin 11 receptor, alpha; procollagen C-endopeptidase enhancer; frizzled homolog 7 (Drosophila); hypothetical gene BC008967; collagen, type VIII, alpha 1; tenascin C (hexabrachion); iroquois homeobox protein 5; hephaestin; integrin, beta 8; synaptic vesicle glycoprotein 2; neuroblastoma, suppression of tumorigenicity 1; insulin-like growth factor binding protein 2, 36 kDa; Homo sapiens cDNA FLJ12280 fis, clone MAMMA1001744; cytokine receptor-like factor 1; potassium intermediate/small conductance calcium-activated channel, subfamily N, member 4; integrin, beta 7; transcriptional co-activator with PDZ-binding motif (TAZ); sine oculis homeobox homolog 2 (Drosophila); KIAA1034 protein; vesicle-associated membrane protein 5 (myobrevin); EGF-containing fibulin-like extracellular matrix protein 1; early growth response 3; distal-less homeobox 5; hypothetical protein FLJ20373; aldo-keto reductase family 1, member C3 (3-alpha hydroxysteroid dehydrogenase, type II); biglycan; transcriptional co-activator with PDZ-binding motif (TAZ); fibronectin 1; proenkephalin; integrin, beta-like 1 (with EGF-like repeat domains); Homo sapiens mRNA full length insert cDNA clone EUROIMAGE 1968422; EphA3; KIAA0367 protein; natriuretic peptide receptor C/guanylate cyclase C (atrionatriuretic peptide receptor C); hypothetical protein FLJ14054; Homo sapiens mRNA; cDNA DKFZp564B222 (from clone DKFZp564B222); BCL2/adenovirus E1B 19 kDa interacting protein 3-like; AE binding protein 1; and cytochrome c oxidase subunit VIIa polypeptide 1 (muscle). In addition, these isolated human umbilicus-derived cells express a gene for each of interleukin 8; reticulon 1; chemokine (C—X—C motif) ligand 1 (melonoma growth stimulating activity, alpha); chemokine (C—X—C motif) ligand 6 (granulocyte chemotactic protein 2); chemokine (C—X—C motif) ligand 3; and tumor necrosis factor, alpha-induced protein 3, wherein the expression is increased relative to that of a human cell which is a fibroblast, a mesenchymal stem cell, an iliac crest bone marrow cell, or placenta-derived cell. The cells are capable of self-renewal and expansion in culture, and have the potential to differentiate into cells of other phenotypes.

Methods of deriving cord tissue mesenchymal stem cells from human umbilical tissue are provided. The cells are capable of self-renewal and expansion in culture, and have the potential to differentiate into cells of other phenotypes. The method comprises (a) obtaining human umbilical tissue; (b) removing substantially all of blood to yield a substantially blood-free umbilical tissue, (c) dissociating the tissue by mechanical or enzymatic treatment, or both, (d) resuspending the tissue in a culture medium, and (e) providing growth conditions which allow for the growth of a human umbilicus-derived cell capable of self-renewal and expansion in culture and having the potential to differentiate into cells of other phenotypes.

Tissue can be obtained from any completed pregnancy, term or less than term, whether delivered vaginally, or through other routes, for example surgical Cesarean section. Obtaining tissue from tissue banks is also considered within the scope of the present invention.

The tissue is rendered substantially free of blood by any means known in the art. For example, the blood can be physically removed by washing, rinsing, and diluting and the like, before or after bulk blood removal for example by suctioning or draining. Other means of obtaining a tissue substantially free of blood cells might include enzymatic or chemical treatment.

Dissociation of the umbilical tissues can be accomplished by any of the various techniques known in the art, including by mechanical disruption, for example, tissue can be aseptically cut with scissors, or a scalpel, or such tissue can be otherwise minced, blended, ground, or homogenized in any manner that is compatible with recovering intact or viable cells from human tissue.

In a presently preferred embodiment, the isolation procedure also utilizes an enzymatic digestion process. Many enzymes are known in the art to be useful for the isolation of individual cells from complex tissue matrices to facilitate growth in culture. As discussed above, a broad range of digestive enzymes for use in cell isolation from tissue is available to the skilled artisan. Ranging from weakly digestive (e.g. deoxyribonucleases and the neutral protease, dispase) to strongly digestive (e.g. papain and trypsin), such enzymes are available commercially. A nonexhaustive list of enzymes compatable herewith includes mucolytic enzyme activities, metalloproteases, neutral proteases, serine proteases (such as trypsin, chymotrypsin, or elastase), and deoxyribonucleases. Presently preferred are enzyme activites selected from metalloproteases, neutral proteases and mucolytic activities. For example, collagenases are known to be useful for isolating various cells from tissues. Deoxyribonucleases can digest single-stranded DNA and can minimize cell-clumping during isolation. Enzymes can be used alone or in combination. Serine protease are preferably used in a sequence following the use of other enzymes as they may degrade the other enzymes being used. The temperature and time of contact with serine proteases must be monitored. Serine proteases may be inhibited with alpha 2 microglobulin in serum and therefore the medium used for digestion is preferably serum-free. EDTA and DNase are commonly used and may improve yields or efficiencies. Preferred methods involve enzymatic treatment with for example collagenase and dispase, or collagenase, dispase, and hyaluronidase, and such methods are provided wherein in certain preferred embodiments, a mixture of collagenase and the neutral protease dispase are used in the dissociating step. More preferred are those methods which employ digestion in the presence of at least one collagenase from Clostridium histolyticum, and either of the protease activities, dispase and thermolysin. Still more preferred are methods employing digestion with both collagenase and dispase enzyme activities. Also preferred are methods which include digestion with a hyaluronidase activity in addition to collagenase and dispase activities. The skilled artisan will appreciate that many such enzyme treatments are known in the art for isolating cells from various tissue sources. For example, the LIBERASE BLENDZYME (Roche) series of enzyme combinations of collagenase and neutral protease are very useful and may be used in the instant methods. Other sources of enzymes are known, and the skilled artisan may also obtain such enzymes directly from their natural sources. The skilled artisan is also well-equipped to assess new, or additional enzymes or enzyme combinations for their utility in isolating the cells of the invention. Preferred enzyme treatments are 0.5, 1, 1.5, or 2 hours long or longer. In other preferred embodiments, the tissue is incubated at 37.degree. C. during the enzyme treatment of the dissociation step. Diluting the digest may also improve yields of cells as cells may be trapped within a viscous digest.

While the use of enzyme activites is presently preferred, it is not required for isolation methods as provided herein. Methods based on mechanical separation alone may be successful in isolating the instant cells from the umbilicus as discussed above.

The cells can be resuspended after the tissue is dissociated into any culture medium as discussed herein above. Cells may be resuspended following a centrifugation step to separate out the cells from tissue or other debris. Resuspension may involve mechanical methods of resuspending, or simply the addition of culture medium to the cells.

Providing the growth conditions allows for a wide range of options as to culture medium, supplements, atmospheric conditions, and relative humidity for the cells. A preferred temperature is 37.degree. C., however the temperature may range from about 35.degree. C. to 39.degree. C. depending on the other culture conditions and desired use of the cells or culture.

Presently preferred are methods which provide cells which require no exogenous growth factors, except as are available in the supplemental serum provided with the Growth Medium. Also provided herein are methods of deriving umbilical cells capable of expansion in the absence of particular growth factors. The methods are similar to the method above, however they require that the particular growth factors (for which the cells have no requirement) be absent in the culture medium in which the cells are ultimately resuspended and grown in. In this sense, the method is selective for those cells capable of division in the absence of the particular growth factors. Preferred cells in some embodiments are capable of growth and expansion in chemically-defined growth media with no serum added. In such cases, the cells may require certain growth factors, which can be added to the medium to support and sustain the cells. Presently preferred factors to be added for growth on serum-free media include one or more of FGF, EGF, IGF, and PDGF. In more preferred embodiments, two, three or all four of the factors are add to serum free or chemically defined media. In other embodiments, LIF is added to serum-free medium to support or improve growth of the cells.

Methods are provided wherein the cells can undergo at least 25, 30, 35, or 40 doublings prior to reaching a senescent state. Methods for deriving cells capable of doubling to reach 10.sup.14 cells or more are provided. Preferred are those methods which derive cells that can double sufficiently to produce at least about 10.sup.14, 10.sup.15, 10.sup.16, or 10.sup.17 or more cells when seeded at from about 10.sup.3 to about 10.sup.6 cells/cm.sup.2 in culture. Preferably these cell numbers are produced within 80, 70, or 60 days or less. In one embodiment, cord tissue mesenchymal stem cells are isolated and expanded, and possess one or more markers selected from a group comprising of CD10, CD13, CD44, CD73, CD90, CD141, PDGFr-alpha, or HLA-A,B,C. In addition, the cells do not produce one or more of CD31, CD34, CD45, CD117, CD141, or HLA-DR, DP, DQ.

In one embodiment, bone marrow MSC lots are generated, means of generating BM MSC are known in the literature and examples are incorporated by reference.

In one embodiment BM-MSC are generated as follows

1. 500 mL Isolation Buffer is prepared (PBS+2% FBS+2 mM EDTA) using sterile components or filtering Isolation Buffer through a 0.2 micron filter. Once made, the Isolation Buffer was stored at 2-8.degree. C.
2. The total number of nucleated cells in the BM sample is counted by taking 10 .mu.L BM and diluting it 1/50-1/100 with 3% Acetic Acid with Methylene Blue (STEMCELL Catalog #07060). Cells are counted using a hemacytometer.
3. 50 mL Isolation Buffer is warmed to room temperature for 20 minutes prior to use and bone marrow was diluted 5/14 final dilution with room temperature Isolation Buffer (e.g. 25 mL BM was diluted with 45 mL Isolation Buffer for a total volume of 70 mL).
4. In three 50 mL conical tubes (BD Catalog #352070), 17 mL Ficoll-Paque™ PLUS (Catalog #07907/07957) is pipetted into each tube. About 23 mL of the diluted BM from step 3 was carefully layered on top of the Ficoll-Paque™ PLUS in each tube.
5. The tubes are centrifuged at room temperature (15-25.degree. C.) for 30 minutes at 300.times.g in a bench top centrifuge with the brake off.
6. The upper plasma layer is removed and discarded without disturbing the plasma:Ficoll-Paque™ PLUS interface. The mononuclear cells located at the interface layer are carefully removed and placed in a new 50 mL conical tube. Mononuclear cells are resuspended with 40 mL cold (2-8.degree. C.) Isolation Buffer and mixed gently by pipetting.
7. Cells were centrifuged at 300.times.g for 10 minutes at room temperature in a bench top centrifuge with the brake on. The supernatant is removed and the cell pellet resuspended in 1-2 mL cold Isolation Buffer.
8. Cells were diluted 1/50 in 3% Acetic Acid with Methylene Blue and the total number of nucleated cells counted using a hemacytometer.
9. Cells are diluted in Complete Human MesenCult®-Proliferation medium (STEMCELL catalog #05411) at a final concentration of 1.times.10.sup.6 cells/mL.
10. BM-derived cells were ready for expansion and CFU-F assays in the presence of GW2580, which can then be used for specific applications.

In one embodiment of the invention, said regeneration of said nucleus pulposus is achieved by administration of a biological treatment. The biological treatment may comprises a biologically active component selected from the following: anti-cytokines; cytokines; anti-interleukin-1 components (anti-IL-1); anti-TNF alpha; growth factors; LIM mineralization proteins; stem cell material, autogenic chondrocytes; allogenic chondrocytes; autogenic chondrocytes with one of a retroviral viral vector or a plasmid viral vector; allogenic chondrocytes with one of a retroviral viral vector or a plasmid viral vector; and fibroblasts.

In one embodiment of the invention, the stem cell material may be from undifferentiated cells, or it may be from cells that have differentiated and have subsequently been returned to their undifferentiated state. Regardless of whether the cells intended for implantation have begun to differentiate before selection for use in a disc space, in some embodiments the stem cell material comprises cells that have been induced to express at least one characteristic of human intervertebral disc cells (such as fibroblast cells, chondrocyte cells, or notochordal cells) before the material is implanted in a disc. Alternatively, undifferentiated stem cell material and a material capable of inducing stem cell differentiation may be combined just prior to, during, or after implantation in a disc space so that the stem cell material differentiates in the disc space to express at least one characteristic of human intervertebral disc cells. In some embodiments, the stem cell material is provided in conjunction with a collagen-based material, which may be a collagen-rich lattice. The collagen-based material may be provided in dehydrated form, and rehydrated after administration, or it may be provided in a hydrated form, such as a slurry or gel. Cross-linking agents such as glutaraldehyde may be included in the collagen-based material to promote collagen crosslinking.

In addition, radio-contrast materials may be included to enhance imaging. Performance-enhancing additives such as analgesics and/or antibiotics may be included to provide additional therapeutic benefits. In some preferred embodiments the stem cell material is provided as a stem cell isolate, which may be substantially free of non-stem cell material. Objects and advantages of the claimed invention will be apparent from the following description.

According to the present invention, stimulation of perispinal angiogenesis may be conducted, and the stem cells may be transplanted into the nucleus pulposus cavity of intervertebral disc to provide tropic factors to the surrounding cells in the transplanted portion when transplanted. Further, the stem cells per se are induced to differentiate due to the tropic factors derived from the surrounding tissue and differentiation inducing factors etc. If these stem cells are transplanted into a degenerated intervertebral disc, they are induced to cells exhibiting the morphology of intervertebral disc cells and can regenerate intervertebral disc tissue. As such stem cells, it is possible to use those derived from the subject individual or homogenous or heterogenous individuals. Specifically, mesenchymal stem cells, multipotent stem cells, etc. may be mentioned. The collected stem cells may be directly suspended into our developed medium for stem cells of intervertebral disc regeneration or embedded in a cell carrier (for example, agarose, alginate, atelocollagen, etc.) for transplant. However, according to our experiments, the method of culturing and causing to proliferate the cells in our developed medium for stem cells in regeneration of intervertebral disc, then suspending them in the above medium or embedding them in a cell carrier and transplanting them into the nucleus pulposus cavity of intervertebral discs is preferable in that it has a high effect of regeneration of intervertebral discs. As the medium for stem cells for intervertebral disc regeneration according to the present invention, it is possible to use a commercially available culture solution used in general for culturing mesenchymal stem cells, that is, a stem cell medium into which previously synthesized growth factors and serum derived from other animals are mixed. However, we conducted experiments using the following medium from the viewpoint that it is desirable to transplant cells in such a state as close as possible to the biological material receiving the transplant when transplanting stem cells in vivo and succeeded in causing the regeneration of intervertebral discs by transplanting mesenchymal stem cells into the nucleus pulposus cavity of intervertebral discs. That is, first, we collected the necessary amount of whole blood from the individual for use in the transplant and used a centrifuge to separate the blood cells to thereby obtain the serum. We sterilized this serum using a sterilization filter and, followed by heating in a thermostatic tank at, for example, 50 to 70.degree. C., preferably 55 to 60.degree. C., for, for example, 20 to 40 minutes, preferably 25 to 35 minutes, to immobilize it. Then, we injected this into a previously sterilized medium for cell culture, for example, DMEM (Dulbecco's Modified Eagle Medium), DMEM/F-12MEM (Minimum Essential Medium), RPMI1640, BME (Basal Medium Eagle), Brinster's BMOC-3, BGJb, CMRL 1066, F-10, F-12, Glasgow MEM, IMDM (Iscove's Dulbecco's Medium), McCoy's5A Medium, MCDB131 Medium, Medium 199, NCTC-109 Medium, Waymouth's MB 752-1Medium, William's Medium E, Opti-MEM I Reduced-Serum Medium, or another cell culture medium so that said autologous plasma became a concentration of 1 to 25% by weight, preferably 5 to 20% by weight. Note that these media have been known from the past, as cell culture media, and are commercially available. These media can be used alone or in any mixture thereof. According to the present invention, further, an antibiotic may be added to the medium in such a concentration that an antibacterial action can be provided and that the cultured cells can be survived, specifically for example, penicillin in 8,000 to 10,000 U/ml, streptomycin in 8,000 to 10,000 .mu.g/ml, amphotericin B in 20 to 25 .mu.g/ml, gentamycin in 0.5 to 50 .mu.g/ml, hygromycin B in 25 to 1000 .mu.g/ml, kanamycin sulfate in 0.5 to 50 .mu.g/ml, actinomycin D in 0.5 to 50 .mu.g/ml, neomycin sulfate in 8,000 to 10,000 .mu.g/ml, etc. alone or in any mixture thereof so as to obtain the medium for stem cell in the regeneration of intervertebral disc.

The method of transplant of the stem cells into the intervertebral disc tissue is not particularly limited, but may be carried out by exposing the intervertebral disc, then using a syringe or another suitable means capable of injecting a liquid or gel-like cell carrier or, when using a scaffold (e.g., a bioabsorptive polymer etc.), directly placing the same in the intervertebral disc. In the case of human subjects, sometimes the intervertebral disc is cracked or has holes. In this case, to prevent leakage of the transplanted cells, a known conventional adhesive for biological tissue (e.g., fibrin) or periorsteum or another connective tissue is used for repair. This transplant method is used for the purpose of suppressing degeneration of intervertebral disc or regenerating an intervertebral disc at the time of surgery having a direct or indirect effect on the intervertebral disc (e.g., intervertebral disc herniotomy, etc.) Further, for non-surgically treated intervertebral disc herniation, lumbar spondylolisthesis and lumbar spondylosis deformans as well, when intervertebral disc degeneration is progressed, it can be suppressed or the disc regenerated by use of the present medium for stem cell in intervertebral disc regeneration and intervertebral disc regeneration method. In these cases, it is desirable that the transplanted stem cells used be autogenous, but cells obtained from homogenous or heterogenous individuals may also be used.

As used herein, a “biological treatment” includes but is not limited to a “biologically active component”, with or without a “biological additive”.

A “biologically active component” includes but is not limited to anti-cytokines; cytokines; anti-interleukin-1 components (anti-IL-1); anti-TNF alpha; “growth factors”; LIM mineralization proteins; “stem cell material”; autogenic chondrocytes; allogenic chondrocytes, such as those described in U.S. Patent Application Publication No. 2005/0196387, the entire disclosure of which is incorporated herein by reference; autogenic chondrocytes with retroviral viral vector or plasmid viral vector; allogenic chondrocytes with retroviral viral vector or plasmid viral vector; and fibroblasts. The acronym “LIM” is derived from the three genes in which the LIM domain was first described. The LIM domain is a cysteine-rich motif defined by 50-60 amino acids with the consensus sequence CX.sub.2CX.sub.16-23HX.sub.2CX.sub.2CX.sub.2CX.sub.16-21CX.sub.2-(C/H/D), which contains two closely associated zinc-binding modules. LIM mineralization proteins include but are not limited to those described in U.S. Patent Application Publication No. 2003/0180266 A1, the disclosure of which is incorporated herein by reference. “Growth factors” include but are not limited to transforming growth factor (TGF)-beta 1, TGF-beta 2, TGF-beta 3, bone morphogenetic protein (BMP)-2, BMP-3, BMP-4, BMP-6, BMP-7, BMP-9, fibroblast growth factor (FGF), platelet derived growth factor (PDGF), insulin-like growth factor (ILGF); human endothelial cell growth factor (ECGF); epidermal growth factor (EGF); nerve growth factor (NGF); and vascular endothelial growth factor (VEGF). “Anti-IL-1” components include but are not limited to those described in U.S. Patent Application Publication Nos. 2003/0220283 and 2005/0260159, the entire disclosures of which are incorporated herein by reference. “Stem cell material” includes but is not limited to dedifferentiated stem cells, undifferentiated stem cells, and mesenchymal stem cells. “Stem cell material” also includes but is not limited to stem cells extracted from marrow, which may include lipo-derived stem cell material and adipose-derived stem cell material, such as described in U.S. Publication Nos. 2004/0193274 and 2005/0118228, each of which is incorporated herein by reference. “Stem cell material” also includes but is not limited to stem cells derived from adipose tissue as described in U.S. Patent Application Publication Nos. 2003/0161816, 2004/0097867 and 2004/0106196, each of which is incorporated herein by reference. Furthermore, said “biologically active component” also includes but is not limited to cartilage derived morphogenetic protein (CDMP); cartilage inducing factor (CIP); proteoglycans; hormones; and matrix metalloproteinases (MMP) inhibitors, which act to inhibit the activity of MMPs, to prevent the MMPs from degrading the extracellular matrix (ECM) produced by cells within the nucleus pulposus of the disc. Exemplary MMP inhibitors include but are not limited to tissue inhibitors, such as TIMP-1 and TIMP-2. Certain MMP inhibitors are also described in U.S. Patent Application Publication No. 2004/0228853, the entire disclosure of which is incorporated herein by reference.

As used herein, a “biological additive” includes but is not limited to “biomaterial carriers”, “therapeutic agents”, “liquids” and “lubricants.”

“Biomaterial carriers” include but are not limited to collagen, gelatin, hyaluronic acid, fibrin, albumin, keratin, silk, elastin, glycosaminoglycans (GAGs), polyethylene glycol (PEG), polyethylene oxide (PEO), polyvinyl alcohol (PVA) hydrogel, polyvinyl pyrrolidone (PVP), co-polymers of PVA and PVP, other polysaccharides, platelet gel, peptides, carboxymethyl cellulose, and other modified starches and celluloses. Collagen includes but is not limited to collagen-based material, which may be autogenic, allogenic, xenogenic or of human-recombinant origin, such as the collagen-based material described in U.S. Patent Application Publication Nos. 2004/0054414 and 2004/0228901, the entire disclosures of which are incorporated herein by reference.

“Therapeutic agents” include but are not limited to nutrients, analgesics, antibiotics, anti-inflammatories, steroids, antiviricides, vitamins, amino acids and peptides. Nutrients include but are not limited to substances that promote disc cell survival, such as glucose and pH buffers, wherein the pH buffer provides a basic environment in the disc space, which preferably will be a pH of about 7.4. Analgesics include but are not limited to hydrophilic opoids, such as codeine, prodrugs, morphine, hydromorphone, propoxyphene, hydrocodone, oxycodone, meperidine and methadone, and lipophilic opoids, such as fentanyl. Antibiotics include but are not limited to erythromycin, bacitracin, neomycin, penicillin, polymyxin B, tetracyclines, viomycin, chloromycetin and streptomycins, cefazolin, ampicillin, azactam, tobramycin, clindamycin and gentamycin.

1. A method of augmenting regeneration of the nucleus pulposus comprising the steps of: a) identifying a patient with lower back pain; b) assessing perfusion rate of perispinal area of said patient; c) selecting patients possessing a perfusion defect in said lower back; d) administering to said patients an angiogenesis stimulating treatment in the perispinal area at a concentration and frequency sufficient to enhance local perfusion; and e) administering a regenerative intervention intradiscally to the nucleus pulposus.

2. The method of claim 1, wherein said perfusion rate is identified by an imaging means.

3. The method of claim 2, wherein said imaging means is selected from a group comprising of: a) MRI; b) PET-MRI; c) CT-Scan; and d) angiography.

4. The method of claim 1, wherein said angiogenesis stimulating treatment is a stem cell.

5. The method of claim 4, wherein said stem cell is a mesenchymal stem cell.

6. The method of claim 5, wherein said mesenchymal stem cell is derived from the bone marrow.

7. The method of claim 5, wherein said mesenchymal stem cell is derived from Wharton's Jelly.

8. The method of claim 5, wherein said mesenchymal stem cell is derived from the endometrium.

9. The method of claim 5, wherein said mesenchymal stem cell is derived from cord blood.

10. The method of claim 5, wherein said mesenchymal stem cell is derived from placental tissue.

11. The method of claim 5, wherein said mesenchymal stem cell expresses markers selected from a group comprising of; a) CD90; b) CD105; and c) CD73.

12. The method of claim 5, wherein said mesenchymal stem cell does not express markers selected from a group comprising of; a) CD14; b) CD34; and c) HLA II.

13. The method of claim 5, wherein said mesenchymal stem cell is hypoxia treated.

14. The method of claim 5, wherein said mesenchymal stem cell is treated with a histone deacetylase inhibitor.

15. The method of claim 14, wherein said histone deacetylase inhibitor is valproic acid.

16. The method of claim 1, wherein said regeneration of said nucleus pulposus is achieved by administration of a biological treatment.

17. The method of claim 16, wherein said biological treatment comprises a biologically active component.

18. The method of claim 17 wherein the biological treatment comprises a biologically active component selected from anti-cytokines; cytokines; anti-interleukin-1 components (anti-IL-1); anti-TNF alpha; growth factors; LIM mineralization proteins; stem cell material, autogenic chondrocytes; allogenic chondrocytes; autogenic chondrocytes with one of a retroviral viral vector or a plasmid viral vector; allogenic chondrocytes with one of a retroviral viral vector or a plasmid viral vector; and fibroblasts.

19. The method of claim 17 wherein the biological treatment comprises a biologically active component selected from transforming growth factors, bone morphogenetic proteins, fibroblast growth factors, platelet derived growth factor (PDGF), insulin-like growth factor (ILGF); human endothelial cell growth factor (ECGF); epidermal growth factor (EGF); nerve growth factor (NGF); and vascular endothelial growth factor (VEGF).

20. The method of claim 17 wherein the biological treatment comprises stem cell material selected from dedifferentiated stem cells, undifferentiated stem cells, mesenchymal stem cells, marrow-extracted stem cell material and adipose-derived stem cell material.
